(19) United States
(12) Patent Application Publication
(10) Pub. No.: US 2010/0114016 A1
(43) Pub. Date: May 6, 2010

(54) Device and Method for Washing Nasal Passages of Children

(75) Inventors: David Gallo, San Diego, CA (US); Diane Heatley, Madison, WI (US); Jeff Harlan, Corona, CA (US)

Correspondence Address: PROCOPIO, CORY, HARGREAVES & SAVITCH LLP, 530 B STREET, SUITE 2100, SAN DIEGO, CA, 92101, US

(73) Assignee: Med-Systems, Inc., Madison, WI (US)

(21) Appl. No.: 12/604,217

(22) Filed: Oct. 22, 2009

Related U.S. Application Data
(60) Provisional application No. 61/109,656, filed on Oct. 30, 2008.

(51) Int. Cl. A61M 31/00

(52) U.S. Cl. 604/73

(57) ABSTRACT

A method of using a nasal wash system includes providing a nasal washing device including a container including a saline solution, a cap for the container, a delivery tube carried by the cap for withdrawing saline solution from the container, an atomizing nozzle for spraying the saline solution, a conduit carrying the atomizing nozzle, the conduit including a malleable portion, and a pump mechanism coupled to the malleable conduit and including a movable pump member; adjusting the malleable portion of the conduit to a desired configuration to control a point of impact of a discharge mist from the atomizing nozzle; and moving the movable pump member of the pump mechanism so that saline solution is withdrawn from the container through the delivery tube, through the pump mechanism, through the conduit, and sprayed out of the atomizing nozzle as a discharge mist, while the atomizing nozzle remains stationary.

### CROSS-REFERENCE TO RELATED APPLICATION

This application claims the benefit of provisional patent application 61/109,656, filed Oct. 30, 2008 under 35 U.S.C. 119(e). This provisional patent application is incorporated by reference herein as though set forth in full.

### FIELD OF THE INVENTION

The present invention is in the field of devices and methods for washing nasal passages of children.

### BACKGROUND OF THE INVENTION

Nasal washes have been used in the past to deliver a saline solution to an adult user's nasal passages (through a user's nostrils) to wash or lavage the nasal passages to relieve symptoms caused by sinus infections, allergies and the common cold, including nasal congestion, irritated nasal passages, nasal drainage, post-nasal drip, cough, and nasal headaches. A number of devices have been developed for delivering a saline solution to the nasal passages of a child, but they all suffer from a number drawbacks.

### SUMMARY OF THE INVENTION

The present invention involves a nasal wash system and method that provide an all-natural, scientifically proven, effective method for relieving nasal congestion and soothing irritated nasal passages of a child's nose. Using a gentle saline solution to wash or lavage the nasal passages of the child's nose thins mucus buildup in the nose and washes away this mucus, improving breathing. The nasal wash system and method allow parents to ease their infants' and children's nasal discomfort, congestion, allergies, sinus headaches, and runny noses. Some of the benefits of the nasal wash system and method to children include that the nasal wash system and method use an ultra-fine mist for a child's sensitive nose, the saline solution uses 100% all-natural ingredients, the saline solution is preservative free, the nasal wash system and method use pre-measured and precise doses, one-way, anti-back wash valve(s) that prevent re-contamination, and the nasal wash system and method are safe for newborns to children (e.g., up to 12 years old or older).

Another aspect of the invention involves a method of using a nasal wash system. The method includes providing a nasal washing device including a container including a saline solution, a cap for the container, a delivery tube carried by the cap for withdrawing saline solution from the container, an atomizing nozzle for spraying the saline solution, a conduit carrying the atomizing nozzle, the conduit including a malleable portion, and a pump mechanism coupled to the malleable conduit and including a movable pump member; adjusting the malleable portion of the conduit to a desired configuration to control a point of impact of a discharge mist from the atomizing nozzle; and moving the movable pump member of the pump mechanism so that saline solution is withdrawn from the container through the delivery tube, through the pump mechanism, through the conduit, and sprayed out of the atomizing nozzle as a discharge mist, while the atomizing nozzle remains stationary.

One or more implementations of the aspect of the invention described immediately above include one or more of the following: the pump mechanism includes a movable annular flange and a fixed pump head, and the annular flange moves downward relative to the fixed pump head to withdraw saline solution from the container through the delivery tube with atomizing nozzle remaining stationary with movement of the movable annular flange, and moving the movable pump member includes moving the annular flange; providing saline solution in a separate saline solution vial with a tear-off top, removing the cap from the container, tearing off the tear-off top from the saline solution vial, dispensing saline solution from the separate saline solution vial into the container, and adding the cap to the container; the saline solution vial includes upper ends with a narrow tube nozzle that the saline solution is dispensed through projecting between the upper ends, the tear-off top includes legs with a gap between the legs that receives the narrow tube nozzle, the lower ends of the legs are attached to the upper ends of the saline solution vial to form tear-off locations where the tear-off top is torn off from the saline solution vial; the method is used for delivering the saline solution to nasal passages of a nose of a child, and the method further including having the child gently blow mucus out of the nose prior to delivering the saline solution to the nasal passages of the nose of the child; the atomizing nozzle includes a tip, and the method further includes applying the only the tip of the atomizing nozzle at a base of a child's nostril without inserting the atomizing nozzle more than ¼ in. into the child's nostril; using one's hand to stabilize a child's head while using the other hand to operate the nasal wash system; spraying an amount of saline mist equivalent to 1-5 pumps of the movable pump member into one of the child's nostrils, and then spraying an amount of saline mist equivalent to 1-5 pumps of the movable pump member into the other nostril of the child; gently blowing the child's nose prior to delivering the saline solution to the nasal passages of the nose of the child; at least one of wiping off with a clean tissue and rinsing with water the atomizing nozzle at least one of prior to delivering the saline solution to the nasal passages of the nose of the child and after delivering the saline solution to the nasal passages of the nose of the child; the method is used for delivering the saline solution to nasal passages of a nose of an infant, and the method further including spraying an amount of saline mist equivalent to one pump of the movable pump member into each nostril of the infant during diaper changes to keep nasal mucus thin and flowing naturally; the method is used for delivering the saline solution to nasal passages of a nose of at least one of a toddler and a preschooler, and the method further includes a) using one's hand to stabilize a child's head while using the other hand to operate the nasal wash system, b) having the child blow nose, c) spraying an amount of saline mist equivalent to multiple pumps of the movable pump member into each nostril, d) repeating steps b and c until nose is clear of mucus; the method is used for delivering the saline solution to nasal passages of a nose of at least one of a school age child and an adolescent, and the method further includes bending the malleable portion of the conduit into a 45-90 degree configuration prior to delivering the saline solution to the nasal passages of the nose, and allowing at least one of a school age child and an adolescent to use the

nasal wash system independently; the saline solution is 100% preservative free saline; the atomizing nozzle includes a one-way valve; the nasal wash system atomizing nozzle delivers a precision measured dose of saline of 0.1 mL/spray; the nasal wash system atomizing nozzle delivering a consistent ultra fine 30-100 micron average particle size mist of saline solution; cleaning the nasal wash system by disassembling the nasal wash system into its components, discarding any remaining saline solution from the container, washing the components in hot, soapy water, and then rinsing the components with clean water; cleaning the nasal wash system by soaking at least one of an assembled atomizing nozzle and a disassembled atomizing nozzle in a vinegar solution, and then air drying at least one of the assembled atomizing nozzle and the disassembled atomizing nozzle; refilling the nasal wash system with saline solution, and then pumping the movable pump member multiple times to eliminate any vinegar solution; mixing saline solution by mixing dry saline solution with water until the dry saline solution ingredients are completely dissolve, and then adding the mixed saline solution to the container.

Further objects and advantages will be apparent to those skilled in the art after a review of the drawings and the detailed description of the preferred embodiments set forth below.

### BRIEF DESCRIPTION OF THE DRAWINGS

FIG. 1 is a front perspective view of a children's nasal wash system constructed in accordance with an embodiment of the invention.

FIG. 2 is a front elevational view of an embodiment of a saline solution vial that may be used with the nasal wash system illustrated in FIG. 1.

FIG. 3 is a bottom plan view of the saline solution vial illustrated in FIG. 1.

FIG. 4 is a top plan view of the saline solution vial illustrated in FIG. 1.

FIG. 5 is a right side elevational view of the saline solution vial illustrated in FIG. 1, the left side elevational view being a mirror image thereof.

FIG. 6 is a perspective view of a top portion of the vial illustrated in FIG. 1.

FIG. 7 is a front elevational view of the top portion of the vial illustrated in FIG. 1, with a removable top removed from the vial.

FIG. 8 is a top plan view of the nasal wash system illustrated in FIG. 1.

FIG. 9 is a front elevational view of the nasal wash system illustrated in FIG. 1.

FIG. 10 is a left side elevational view of the nasal wash system illustrated in FIG. 1.

FIG. 11 is a bottom plan view of the nasal wash system illustrated in FIG. 1.

FIG. 12 is a right side elevational view of the nasal wash system illustrated in FIG. 1.

FIG. 13 is a front elevational exploded view of the nasal wash system illustrated in FIG. 1.

FIG. 14 is a front elevational view of another embodiment of a nasal wash system.

### DETAILED DESCRIPTION OF PREFERRED EMBODIMENTS

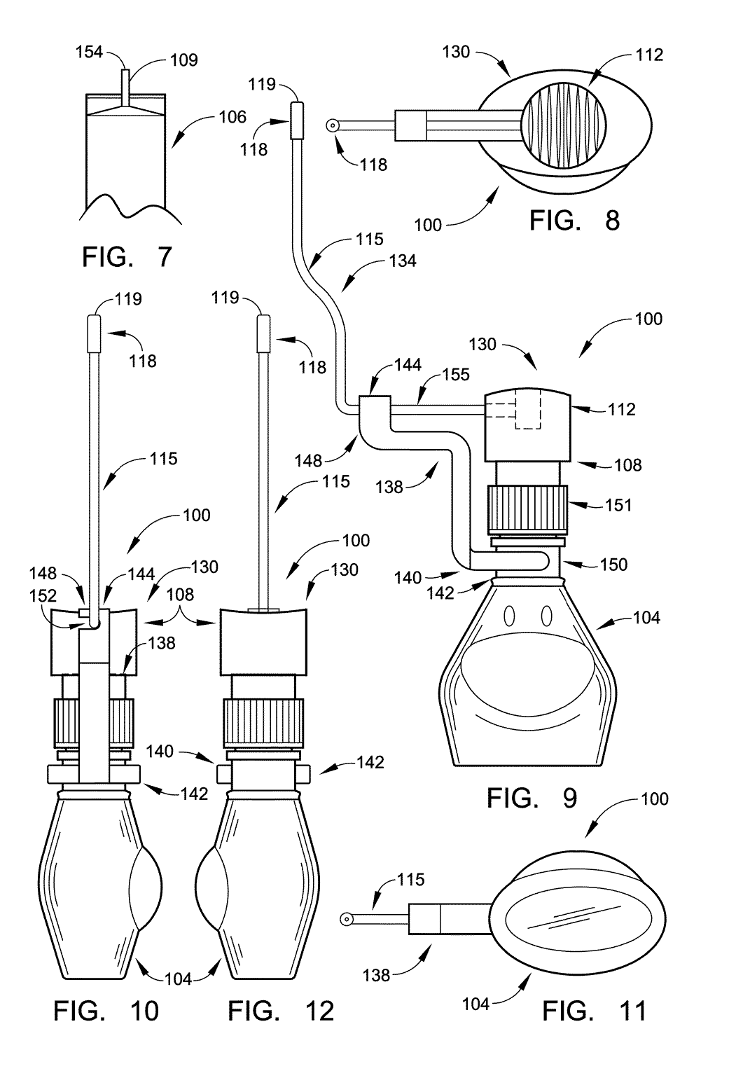

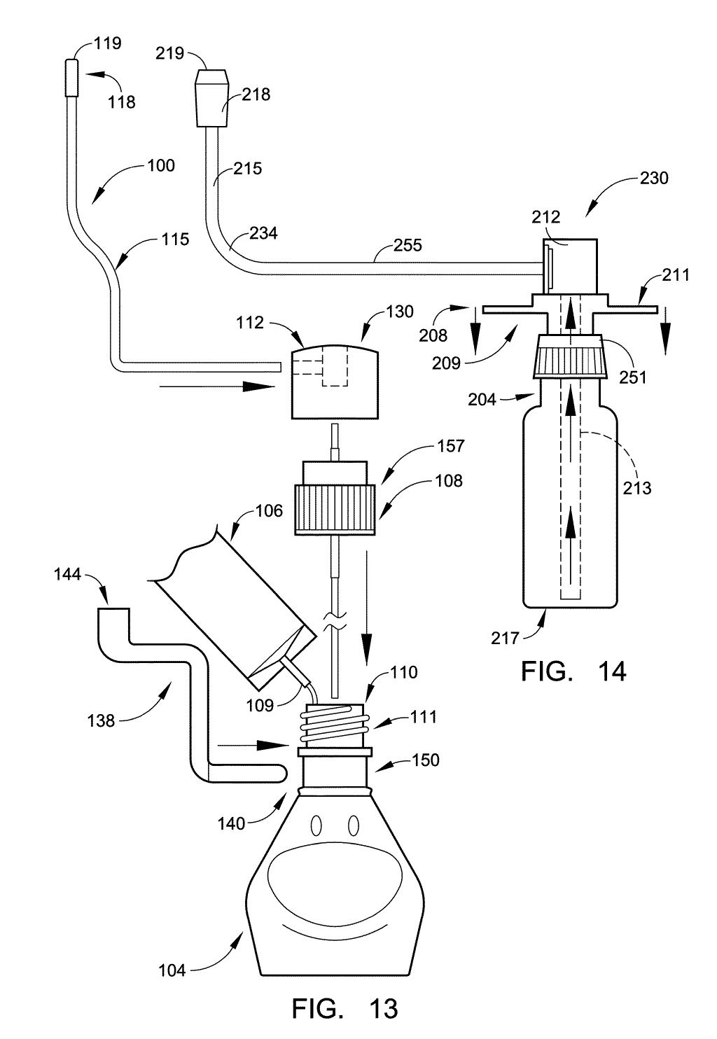

With reference to FIGS. 1-13, an embodiment of a child nasal wash system 100 and an exemplary method of using the child nasal wash system 100 will now be described. The nasal wash system 100 will be described in conjunction with rinsing a child's nasal passages with a saline solution to wash or lavage the nasal passages to relieve symptoms caused by sinus infections, allergies, flu and the common cold, including nasal congestion, irritated nasal passages, nasal drainage, post-nasal drip, cough, and nasal headaches. The child nasal wash system 100 and an exemplary method thin mucus, remove inhaled irritants (e.g., dust, pollen, smoke), promote nasal and sinus drainage, and help reduce swelling of nasal membranes. In alternative embodiments, additionally or alternatively, the nasal wash system 100 is used to wash a tissue of a user (with a saline solution or a different solution) such as, but not by way of limitation, a mucus membrane, eye tissue, and skin or tissue inside an oral cavity.

The nasal wash system 100 includes an atomizer 130 including a pre-mixed preservative-free saline solution. In the embodiment shown, the pre-mixed preservative-free saline solution is supplied in one or more separate saline solution vials 106 including ½ oz of a pre-mixed preservative-free saline solution. The active ingredient in the saline solution is 15 ml of a sodium chloride solution (0.9%). Each vial 106 is substantially cylindrical and includes a removable top 107 that when removed opens/exposes a narrow tube nozzle 109 for dispensing/squirting the pre-mixed preservative-free saline solution through an opening 110 in an externally threaded top 111 of container/bottle 104.

The atomizer 130 includes a conduit 115 for delivering the saline solution to the child's nasal passage(s). At least a distal portion 134 of conduit 115 is malleable (e.g., includes a bendable metal wire therein) to permit a user to define and set a plurality of discharge directions of atomizing nozzle 118 without the conduit 115 from automatically returning to its natural/original orientation. In an alternative embodiment, the conduit 115 is rigid and fixed in a permanent configuration. As illustrated, the nozzle 118 has been oriented for a discharge directed in a substantially vertical direction. It should be noted that operation of pump mechanism 108 requires a displacement of movable pump member in the form of a movable pump head 112. Movable pump head 112 carries conduit 115 and atomizing nozzle 118. Moving pump head 112 can therefore cause a corresponding displacement of nozzle 118, which can undesirably move the point of impact of a discharge mist. The pump mechanism 108 is inherently anti-backwash.

One solution to improve control over a point of impact of a discharge mist is a brace 138. Brace 138 operates to resist motion of nozzle 118 relative to bottle 104. Since bottle 104 is held by a user's hand, nozzle 118 can remain substantially stationary while pump mechanism 108 is actuated. A first end 140 of brace 138 desirably carries attachment structure, generally indicated at 142, to couple the brace 138 to a bottle 104.

A second end 144 of brace 138 carries attachment structure, generally indicated at 148, operable to couple brace 138 to conduit 115. Currently preferred braces 138 are removable, at least at the second end 144, although such is not a requirement.

One convenient location for removable attachment of brace 138 to a bottle 104 is at bottle neck 150. Bottle attachment structure 142 may be formed as a resilient clip-on structure sized to engage bottle neck 150.

A second end 144 of brace 138 carries attachment structure 148 for coupling the brace 138 to a conduit 115. It is currently preferred that the attachment structure 148 forms a removable coupling or connection. The attachment structure 148 includes a channel 152 sized to receive the conduit 115. Channel 152 operates as a motion dampener for nozzle 118 and forms a clip-on attachment to conduit 115. Attachment structure 148 operates as a fulcrum around which a proximal portion 155 of conduit 115 may bend, thereby stabilizing distal portion 134 from rotation and displacement during operation of pump mechanism 108.

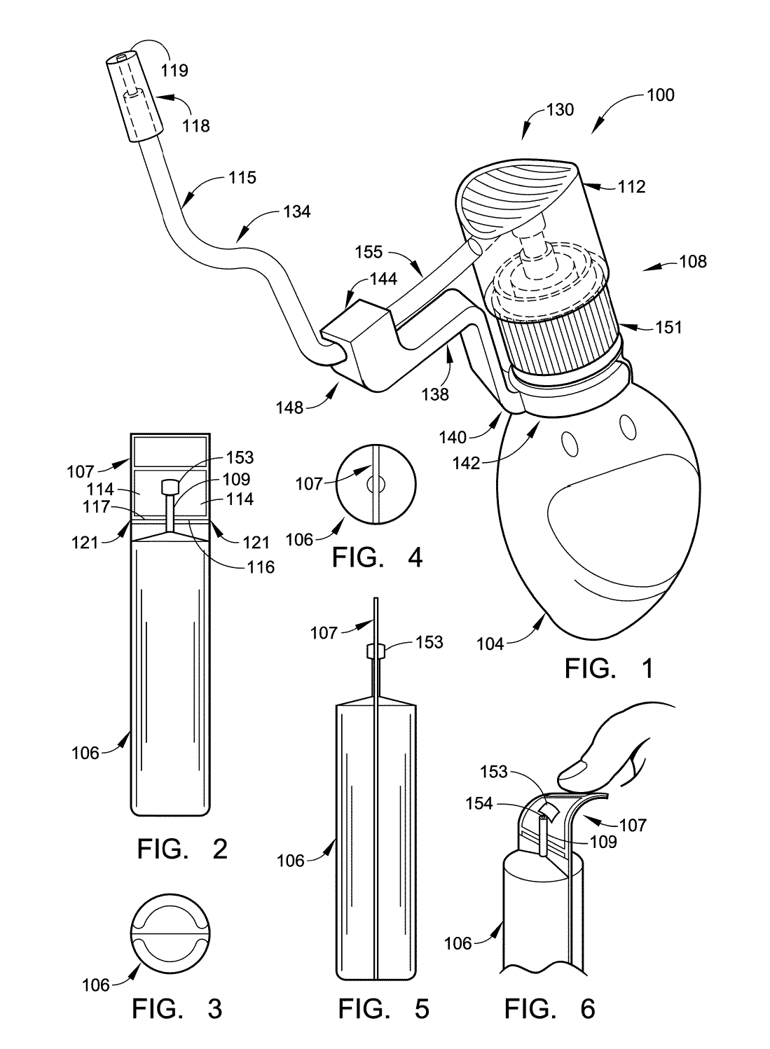

FIG. 14 is an alternative embodiment of an atomizer 230 of the children's nasal wash system 100. Similar to the atomizer 130 of FIG. 1, the atomizer 230 shown in FIG. 2 improves control over a point of impact of a discharge mist. Like elements in atomizer 230 to those in atomizer 130 include like reference numbers except starting with a “2” instead of a “1”.

The atomizer 230 includes a pump mechanism 208 with a movable pump member 209. The movable pump member 209 has a generally cylindrical configuration and includes an annular flange 211. The movable pump member 209 moves relative to head 212, delivery tube 213, cap 251, bottle 204, malleable conduit 215, and atomizing nozzle 218 to cause a pumping action in atomizer 230. In one hand, a user applies one's thumb to bottle bottom 217 and applies two fingers to top surface of annular flange 211. The user applies downward pressure to top surface of annular flange 211 in the direction of the downward arrows shown. This causes movable pump member 209 to move downwardly (relative to head 212, delivery tube 213, cap 251, bottle 204, conduit 215, and atomizing nozzle 218) without atomizing nozzle 218 moving with movable pump member 209. This downward movement of movable pump member 209 causes a pumping action so that saline solution is delivered upwardly in delivery tube 213, through pump mechanism 208, through the conduit 215, and out atomizing nozzle 218.

Similar to conduit 115, at least a distal portion 234 of conduit 215 is malleable to permit a user to define a plurality of discharge directions of atomizing nozzle 218. The pump mechanism 208 allows movement of movable pump member 209 without causing a corresponding displacement of nozzle 218 (preventing undesirable movement of the point of impact of the discharge mist). This improves control over a point of impact of a discharge mist from the atomizing nozzle 218. Since head 212 and bottle 204 do not move with movement of movable pump member 209, nozzle 218 does not move. Since bottle 204 is held by a user's hand, nozzle 218 can remain substantially stationary while pump mechanism 208 is actuated. The pump mechanism 208 is inherently anti-backwash.

With reference to FIGS. 1-13, and especially FIG. 13, the nasal wash system 100 will now be described in use in conjunction with atomizer 130 and saline solution vial 106. In another embodiment, atomizer 230 illustrated in FIG. 14 (or another atomizer) may be used.

First, the second end 144 of the brace 138 is detached from the conduit 115 and the first end 140 of the brace is detached from the bottle neck 150. The pump mechanism 108 is then removed from the externally threaded top 111 of the bottle 104 by unscrewing an internally threaded cap 151 of the pump mechanism 108 from the externally threaded top 111. The removable top 107 is generally rectangular and includes legs 114 with a gap there between that receives narrow tube nozzle 109. The legs 114 include lower ends 116 attached to upper ends 117 of the vial 106 at tear-off locations 121. Next, the removable top 107 of the saline solution vial 106 is removed. To remove the removable top 107 from the vial 106, connections (at tear-off locations 121) between the lower ends 116 of the legs 115 and the upper ends 117 of the vial 106 are separated (e.g., torn apart from each other). This causes a small cap/top 153 to be permanently removed from tip opening 154 of narrow tube nozzle 109, allowing saline fluid flow through tip opening 154. In the embodiment shown, the saline solution vial 106 is not reusable. The saline solution in the vial 106 is then transferred (e.g., by tipping and squeezing vial 106 to cause saline solution to squirt into bottle 104 as shown in FIG. 13). The atomizer 130 is then reassembled. The cap 151 of the pump mechanism 108 is screwed onto the externally threaded top 111 of the bottle 104. The first end 140 of the brace is attached to the bottle neck 150 and the second end 144 of the brace 138 is attached to the conduit 115. Then, the pump head 112 is primed by spraying the atomizer 130 three times into the air. If possible, the child's nose should be gently blown to clear out mucus before applying saline solution. Next, with the user holding the bottle 104 with one's thumb or index finger on the pump head 112, only a tip 119 of the atomizing nozzle 118 is placed at the base of the child's nostril (without inserting the atomizing nozzle 118 more than ¼ in. into the child's nostril). With toddlers (ages 1 to 3 years) and preschoolers (ages 2 to 5 years), the user stabilizes the child's head using the user's free hand. Then, the user sprays 1-5 pumps (by pressing down this many times on the pump head 112) of saline mist into one of child's nostrils. Then, the tip 119 of the atomizing nozzle 118 is placed at the base of the child's other nostril and the user sprays 1-5 pumps of saline mist into this nostril. The child's nose is then gently blown or wiped by the user.

After each use and before each use, the atomizing nozzle 118 is wiped off with a clean tissue or is rinsed with water. Because the nasal wash system 100 includes no preservatives, the nasal wash system 100 is emptied, cleaned, and the solution replaced every 7 (seven) days. Each user of the nasal wash system 100 uses his or her own nasal wash system 100 to prevent the spread of germs between family members/people.

For infants (ages 0 years to toddler beginning age (e.g., 1-3 years)), one spray is sprayed into each nostril of the infant during diaper changes to keep nasal mucus thin and flowing naturally. A bulb syringe is not used to suction mucus out of child's nose since this can cause irritation inside the child's nose.

For toddlers and preschoolers, as indicated above, it is best to use one's free hand to steady the child's head when spraying in the nose. If the child is able to blow mucus out of the nose, multiple sprays should be made into each nostril, followed by the child blowing nose. The steps are repeated until the child's nose is clear.

For school age children and adolescents, the conduit 115 is bent to a 45-90 degree angle, allowing the child to use the device independently. Adult supervision is recommended until child can spray nose without assistance.

A method of cleaning the atomizer 130 will be described. The bottle 104 is taken apart by removing cap 151, pump head 112, conduit 115, and nozzle 118 from the bottle 104. The second end 144 of the brace 138 is detached from the conduit 115 and the first end 140 of the brace 138 is detached from the bottle neck 150. The pump mechanism 108 is then removed from the externally threaded top of the bottle 104 by unscrewing the internally threaded cap 151 of the pump mechanism 108 from the externally threaded top of the bottle 104. Then, any remaining solution is discarded and all components of the atomizer 130 are washed in hot, soapy water. The components are then rinsed with clean water.

The separated components of the atomizer 130 (or the assembled atomizer 130) are soaked in vinegar solution for 20 minutes, and the vinegar solution is discarded and the separated components of the atomizer 130 are allowed to air dry. If the assembled atomizer 130 is soaked in vinegar solution, then the atomizer 130 is disassembled before air drying. The vinegar solution is made by boiling 1.5 cups of water, and adding 0.5 cups of white vinegar. The solution is cooled before cleaning the atomizer 130 and should be discarded after use. Next, once dry, the atomizer 130 is refilled (or assembled and then refilled) with fresh saline solution and pumped ten (10) times to eliminate any vinegar solution. The atomizer 130 should not be placed in the dishwasher, should be emptied and cleaned before storage, and solution over 7 days should not be used.

A method of creating a saline solution and refilling the atomizer 130 with the saline solution will now be described. As an alternative to using the pre-mixed saline solution, saline may be mixed by the user. In a clean container, one or more packets of dry saline solution ingredients are mixed with 0.5 cups of water and stirred until the dry saline solution ingredients are completely dissolved. The mixed solution is then poured into the bottle 104 and the atomizer 130 is reassembled. Any remaining mixed solution is discarded. Solution over 7 days is not used and should be discarded.

The nasal wash system 100 and method provide an all-natural, scientifically proven, effective method for relieving nasal congestion and soothing irritated nasal passages. Using a gentle saline solution, it washes away mucus buildup from the nose and improves breathing. The nasal wash system 100 and method allows parents to ease their infants' and children's nasal discomfort, congestion, allergies, sinus headaches and runny noses. Unlike other saline systems, the nasal wash system 100 combines A) 100% all-natural preservative free saline, B) no backwash, C) precise measured dose, and D) fine mist. No other saline system combines preservative free saline, no backwash, precise measured dose, and fine mist. All four together are important as described below:

### A. 100% All-Natural Preservative Free Saline

Preservatives are required for most pre-mixed saline products. Preservatives in saline have been proven to reduce or even eliminate (or even be harmful) the benefits of saline wash. For example, Benzalkonium Chloride (BZCL) is a common preservative used in saline products; however, BZCL has been proven to damage the sensitive lining of a child's nose. The nasal wash system 100 and method uses preservative free saline specially packaged into plastic vials 106. The saline from the vials 106 is then added to the reusable atomizer 118.

### B. No Backwash

Fluid flow/spray out of the pump mechanism 108 and the atomizing nozzle 218 is through one-way valve(s), preventing any type of backwash. Other saline systems are inherently backwash devices since they provide saline out/air in use the same fluid path. These other saline systems that allow backwash become contaminated with bacteria and viruses after a single use.

### C. Measured Dose

The pump mechanism 108 of the nasal wash system 100 and method provides a precision measured dose of saline. This is important, especially for infants, so as not to introduce too much saline into their noses. The nasal wash system 100 and method delivers a consistent, accurate dose every time (0.1 mL/spray) with different recommended dosages for infants, toddlers, and older children.

### D. Fine Mist

The high pressure, low volume, precision measured, no backwash pump mechanism 108 combined with the super fine spray atomizing nozzle 218 provides a consistent ultra fine “mist” of saline solution (30-100 micron average particle size). This allows a low volume of saline to be administered very evenly and relative deeply into the nose very easily without “touching” the inside of the nose (the inside of infants noses are very sensitive and “contact” with anything can easily cause swelling which makes congestion worse instead of better). With the brace 138, the atomizing nozzle 118 does not move with movement of the pump head 112, preventing contact between the atomizing nozzle 118 and the inside of the child's nose. The inside of a child's nose is extremely sensitive so the ultra fine mist is more comfortable on the inside of the child's nose than greater volume flows/droplets.

The above figures may depict exemplary configurations for the invention, which is done to aid in understanding the features and functionality that can be included in the invention. The invention is not restricted to the illustrated architectures or configurations, but can be implemented using a variety of alternative architectures and configurations. Additionally, although the invention is described above in terms of various exemplary embodiments and implementations, it should be understood that the various features and functionality described in one or more of the individual embodiments with which they are described, but instead can be applied, alone or in some combination, to one or more of the other embodiments of the invention, whether or not such embodiments are described and whether or not such features are presented as being a part of a described embodiment. Thus the breadth and scope of the present invention, especially in the following claims, should not be limited by any of the above-described exemplary embodiments.

Terms and phrases used in this document, and variations thereof, unless otherwise expressly stated, should be construed as open ended as opposed to limiting. As examples of the foregoing: the term “including” should be read as mean “including, without limitation” or the like; the term “example” is used to provide exemplary instances of the item in discussion, not an exhaustive or limiting list thereof; and adjectives such as “conventional,” “traditional,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise. Furthermore, although item, elements or components of the disclosure may be described or claimed in the singular, the plural is contemplated to be within the scope thereof unless limitation to the singular is explicitly stated. The presence of broadening words and phrases such as “one or more,” “at least,” “but not limited to” or other like phrases in some instances shall not be read to mean that the narrower case is intended or required in instances where such broadening phrases may be absent.

What is claimed is:

1. A method of using a nasal wash system, comprising:
providing a nasal washing device including a container including a saline solution, a cap for the container, a delivery tube carried by the cap for withdrawing saline solution from the container, an atomizing nozzle for spraying the saline solution, a conduit carrying the atomizing nozzle, the conduit including a malleable portion, and a pump mechanism coupled to the malleable conduit and including a movable pump member;
adjusting the malleable portion of the conduit to a desired configuration to control a point of impact of a discharge mist from the atomizing nozzle;
moving the movable pump member of the pump mechanism so that saline solution is withdrawn from the container through the delivery tube, through the pump mechanism, through the conduit, and sprayed out of the atomizing nozzle as a discharge mist, while the atomizing nozzle remains stationary.

2. The method of claim 1, wherein the pump mechanism includes a movable annular flange and a fixed pump head, and the annular flange moves downward relative to the fixed pump head to withdraw saline solution from the container through the delivery tube with atomizing nozzle remaining stationary with movement of the movable annular flange, and moving the movable pump member includes moving the annular flange.

3. The method of claim 1, further including providing saline solution in a separate saline solution vial with a tear-off top, removing the cap from the container, tearing off the tear-off top from the saline solution vial, dispensing saline solution from the separate saline solution vial into the container, and adding the cap to the container.

4. The method of claim 3, wherein the saline solution vial includes upper ends with a narrow tube nozzle that the saline solution is dispensed through projecting between the upper ends, the tear-off top includes legs with a gap between the legs that receives the narrow tube nozzle, the lower ends of the legs are attached to the upper ends of the saline solution vial to form tear-off locations where the tear-off top is torn off from the saline solution vial.

5. The method of claim 1, wherein the method is used for delivering the saline solution to nasal passages of a nose of a child, and the method further including having the child gently blow mucus out of the nose prior to delivering the saline solution to the nasal passages of the nose of the child.

6. The method of claim 5, wherein the atomizing nozzle includes a tip, and the method further includes applying the only the tip of the atomizing nozzle at a base of a child's nostril without inserting the atomizing nozzle more than ¼ in. into the child's nostril.

7. The method of claim 5, further including using one's hand to stabilize a child's head while using the other hand to operate the nasal wash system.

8. The method of claim 7, further including spraying an amount of saline mist equivalent to 1-5 pumps of the movable pump member into one of the child's nostrils, and then spraying an amount of saline mist equivalent to 1-5 pumps of the movable pump member into the other nostril of the child.

9. The method of claim 8, further including at least one of wiping off with a clean tissue and rinsing with water the atomizing nozzle at least one of prior to delivering the saline solution to the nasal passages of the nose of the child and after delivering the saline solution to the nasal passages of the nose of the child.

10. The method of claim 1, wherein the method is used for delivering the saline solution to nasal passages of a nose of an infant, and the method further including spraying an amount of saline mist equivalent to one pump of the movable pump member into each nostril of the infant during diaper changes to keep nasal mucus thin and flowing naturally.

11. The method of claim 1, wherein the method is used for delivering the saline solution to nasal passages of a nose of at least one of a toddler and a preschooler, and the method further including:
a) using one's hand to stabilize a child's head while using the other hand to operate the nasal wash system,
b) having the child blow nose,
c) spraying an amount of saline mist equivalent to multiple pumps of the movable pump member into each nostril,
d) repeating steps b and c until nose is clear of mucus.

12. The method of claim 1, wherein the method is used for delivering the saline solution to nasal passages of a nose of at least one of a school age child and an adolescent, and the method further includes bending the malleable portion of the conduit into a 45-90 degree configuration prior to delivering the saline solution to the nasal passages of the nose, and allowing at least one of a school age child and an adolescent to use the nasal wash system independently.

13. The method of claim 1, wherein the saline solution is 100% preservative free saline.

14. The method of claim 1, wherein the atomizing nozzle includes a one-way valve.

15. The method of claim 1, further including the nasal wash system atomizing nozzle delivering a precision measured dose of saline of 0.1 mL/spray.

16. The method of claim 1, further including the nasal wash system atomizing nozzle delivering a consistent ultra fine 30-100 micron average particle size mist of saline solution.

17. The method of claim 1, further including cleaning the nasal wash system by disassembling the nasal wash system into its components, discarding any remaining saline solution from the container, washing the components in hot, soapy water, and then rinsing the components with clean water.

18. The method of claim 1, further including cleaning the nasal wash system by soaking at least one of an assembled atomizing nozzle and a disassembled atomizing nozzle in a vinegar solution, and then air drying at least one of the assembled atomizing nozzle and the disassembled atomizing nozzle.

19. The method of claim 18, further including refilling the nasal wash system with saline solution, and then pumping the movable pump member multiple times to eliminate any vinegar solution.

20. The method of claim 1, further including mixing saline solution by mixing dry saline solution with water until the dry saline solution ingredients are completely dissolve, and then adding the mixed saline solution to the container.
